(19) United States
(12) Patent Application Publication
(10) Pub. No.: US 2025/0308661 A1
(43) Pub. Date: Oct. 2, 2025

(54) SYSTEM, APPARATUS AND METHOD FOR DETECTING USAGE OF A DEVICE FROM ITS ELECTRICITY CONSUMPTION AND/OR VIBRATION

(71) Applicants: Davide Viganò, Redmond, WA (US); Maurizio Macagno, Redmond, WA (US); Roberto Cazzaro, Seattle, WA (US); Steven Czarnota, Canton, MI (US)

(72) Inventors: Davide Viganò, Redmond, WA (US); Maurizio Macagno, Redmond, WA (US); Roberto Cazzaro, Seattle, WA (US); Steven Czarnota, Canton, MI (US)

(21) Appl. No.: 19/093,629

(22) Filed: Mar. 28, 2025

Related U.S. Application Data
(60) Provisional application No. 63/571,276, filed on Mar. 28, 2024.

(51) Int. Cl. G16H 20/00; G01H 17/00; G01R 21/133

(52) U.S. Cl. CPC G16H 20/00; G01H 17/00; G01R 21/133

(57) ABSTRACT

Systems, apparatus and methods for detecting usage of a device from its electricity consumption and/or vibration is disclosed. The disclosure provides a way to detect usage via electricity consumption or vibration to identify usage. That data is then sent to a remote monitoring system via Bluetooth and can ultimately be monitored remotely from anywhere using a cloud based system.

### CROSS REFERENCE TO RELATED APPLICATIONS

This application claims the benefit of priority under 37 CFR § 119 to U.S. Provisional Patent Application Ser. No. 63/571,276, filed on Mar. 28, 2024, titled “System and Method for Detecting Usage of a Device from its Electricity Consumption and Vibration,” the disclosure or which is hereby incorporated by reference in its entirety for all purposes.

### FIELD OF THE INVENTION

The present invention relates generally to systems, apparatuses, and methods for monitoring devices. More specifically, it pertains to a system, apparatus and a method that detects and tracks the usage of a therapeutic device based on its electricity consumption and/or vibrational data.

### BACKGROUND OF THE INVENTION

The ability to monitor the operational status and usage patterns of electrically powered devices is essential for various applications, particularly in healthcare. Therapeutic devices often need to be operated for prescribed durations to achieve optimal results. Accurate monitoring ensures adherence to prescribed therapy, facilitates maintenance planning, and enhances device efficiency.

Traditionally, condition monitoring relies on the use of multiple external sensors to track a device's operational status. For example, accelerometers are often used to assess an electric motor's performance based on vibrational analysis. Furthermore, conventional approaches focus primarily on condition-based maintenance rather than providing real-time compliance tracking or adaptive monitoring of usage-specific parameters.

### SUMMARY OF THE INVENTION

The present disclosure introduces a system, apparatus, and method for monitoring the usage of a therapeutic device by collecting and analyzing electrical consumption and/or vibrational data. The system includes a hardware component designed to measure electrical current flow over time (electrical consumption data) and/or detect inertial and acceleration changes over time (vibrational data) in the therapeutic device; a software component that samples and collects periodic data on changes in electrical current flow and/or vibration from the therapeutic device; a mobile application that utilizes signal processing algorithm and/or machine learning to correlate collected electrical consumption and/or vibrational data with known usage patterns for a specific therapeutic device or a category of therapeutic devices; and a remote monitoring dashboard that displays the therapeutic device's real-time electrical consumption and/or vibration data, identifies activity type, and presents environmental parameters related to device usage.

The system is particularly suited for cold therapy devices, wearable rehabilitation equipment, electrical stimulation therapy devices, and compression therapy machines. In a cold therapy device application, for example, the system ensures that prescribed cooling sessions are followed correctly, detecting deviations such as shortened treatment times or improper usage patterns. By leveraging electrical consumption data and vibrational signals, the disclosure provides an efficient, cost-effective alternative to traditional monitoring solutions while ensuring accuracy and compliance in therapeutic treatments.

### DETAILED DESCRIPTION OF THE FIGURES

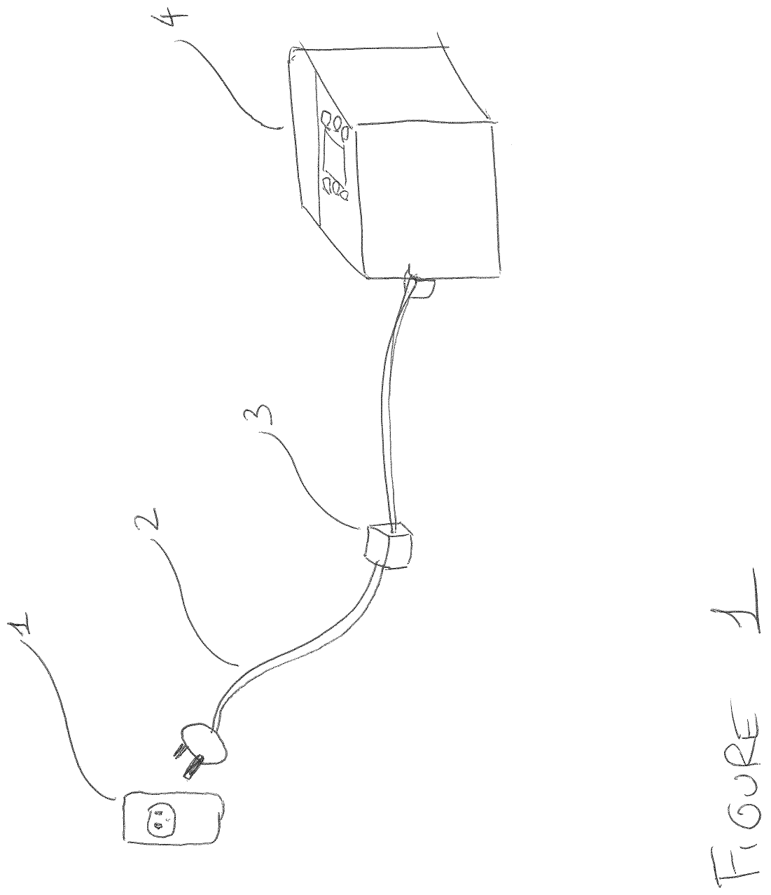

FIG. 1 illustrates an exemplary embodiment of a system for detecting usage of a therapeutic device from its electricity consumption and/or vibration showing the apparatus, device and power connection setup;

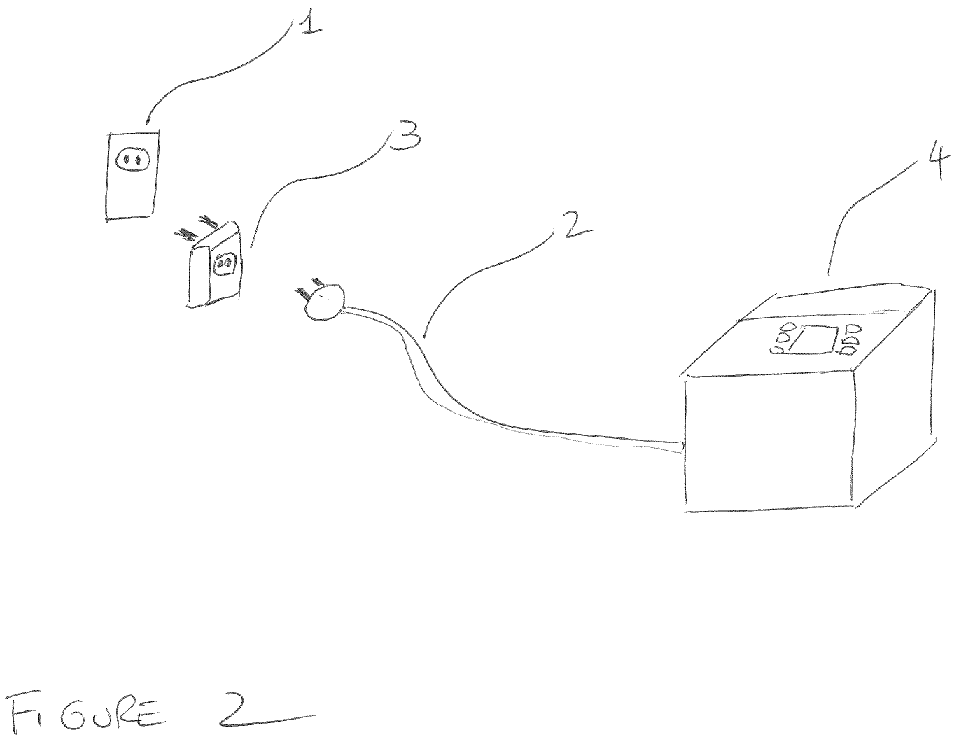

FIG. 2 illustrates another exemplary embodiment of a system for detecting usage of a therapeutic device from its electricity consumption and/or vibration showing the apparatus, device and power connection setup;

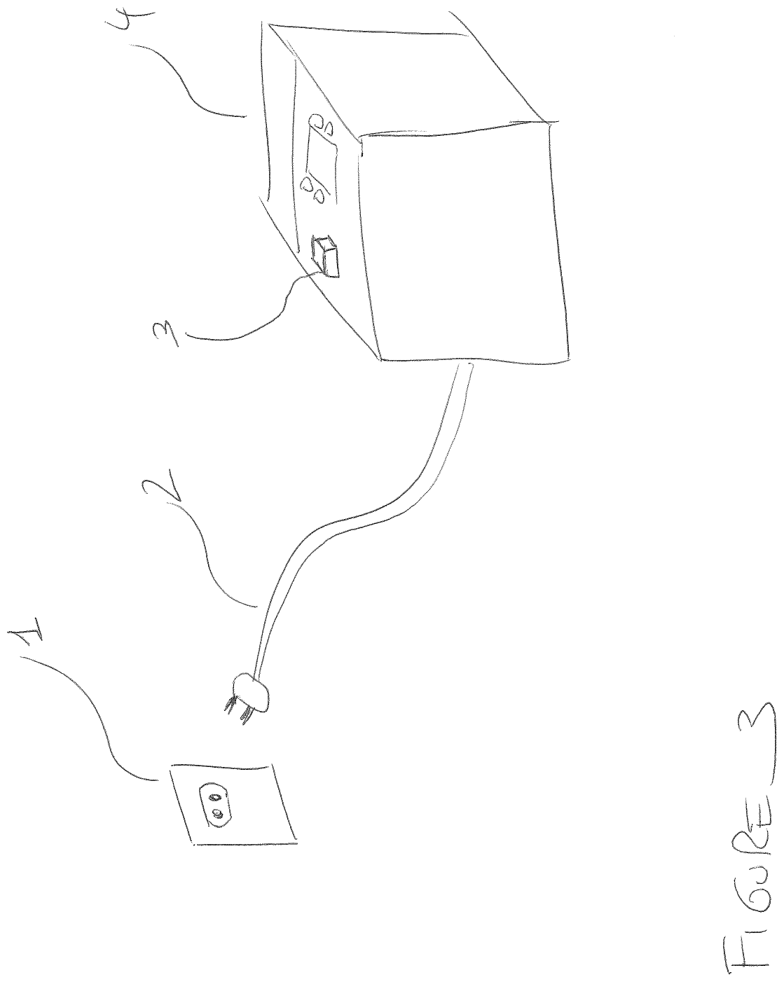

FIG. 3 illustrates another exemplary embodiment of a system for detecting usage of a therapeutic device from its electricity consumption and/or vibration showing the apparatus, device and power connection setup;

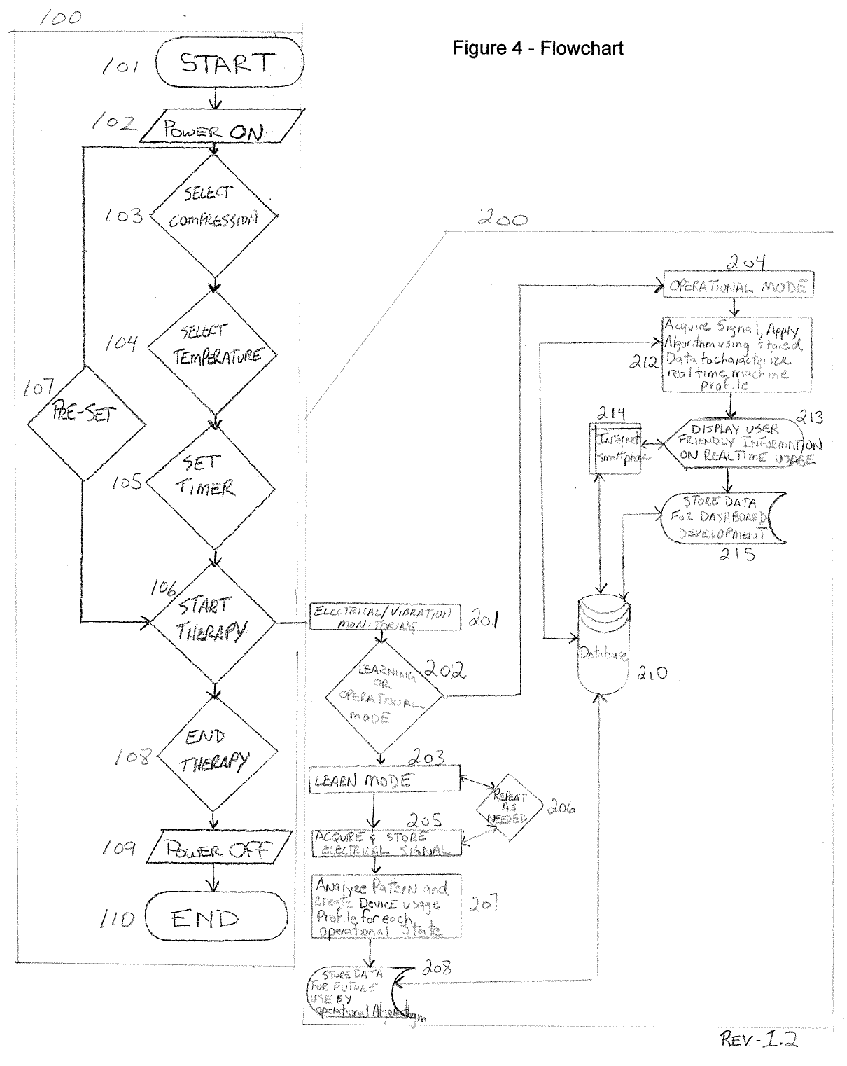

FIG. 4 illustrates an exemplary embodiment of the software components of the system for detecting usage of a therapeutic device from its electricity consumption and/or vibration, which can operate in two primary modes: a learning mode and an operational mode; and

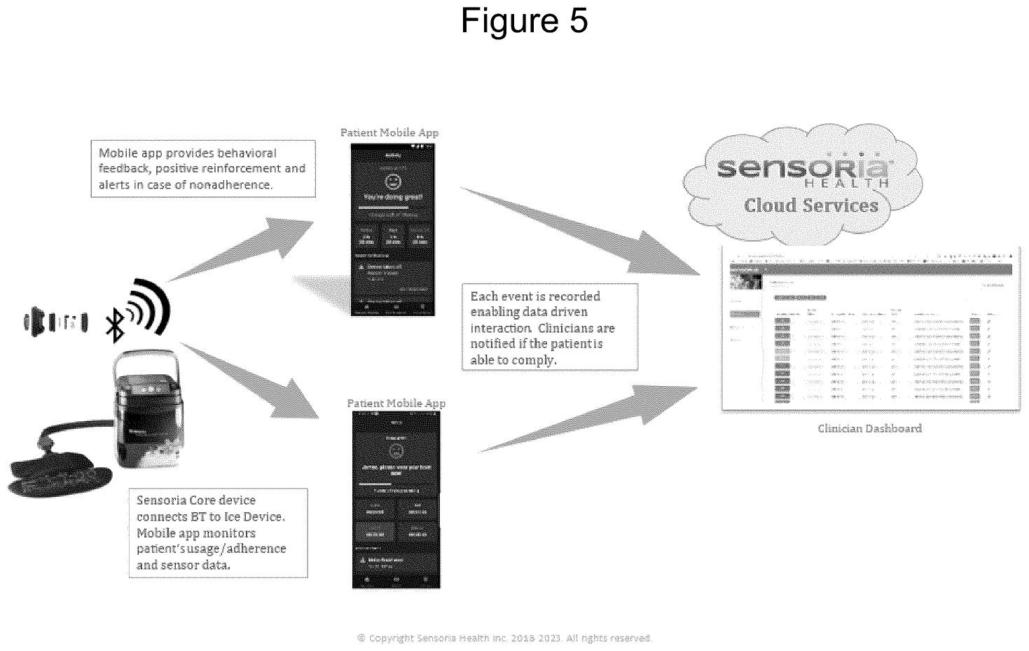

FIG. 5 illustrates an exemplary embodiment of a system for detecting usage of a therapeutic device, e.g., a cold therapy machine, from its electricity consumption and/or vibration.

### DETAILED DESCRIPTION OF THE INVENTION

The disclosure provides a system, an apparatus, and a method that provides an intelligent and non-intrusive method for monitoring the usage of therapeutic devices, ensuring that they are operated correctly and in compliance with prescribed treatment plans. This system is designed to passively track device activity by analyzing electrical consumption and/or vibrational data. If a therapeutic device, such as a cold therapy machine, is prescribed for a specific duration, the system ensures adherence by measuring electrical power draw and vibrational signatures to verify that the device has completed the therapy session. By doing so, the system eliminates the reliance on patient self-reporting and enables healthcare providers to receive accurate, real-time data about therapy adherence and patient compliance.

Unlike traditional methods that require modifications to a therapeutic device's internal components, the hardware component of this disclosure is designed for seamless integration without altering the device's fundamental structure. Depending on the implementation, it may be integrated within a power cord, function as an intermediary adapter, or be attached externally as a wireless sensor module. This flexibility allows it to be used across various medical devices, including compression therapy machines, electrical stimulation therapy units, and wearable rehabilitation systems. The collected data is processed using advanced signal filtering algorithms and analyzed with machine learning models that compare real-time device usage against predefined therapy compliance guidelines. If the system detects irregular behavior, such as incomplete therapy sessions, excessive usage, or power fluctuations, it automatically sends notifications to both the patient and healthcare provider via the mobile application or remote dashboard.

One of the key advantages of this system is its ability to facilitate real-time communication between patients and healthcare providers. The mobile application serves as an interactive user interface, allowing patients to review their session history, receive therapy reminders, and monitor adherence trends over time. If the system identifies potential issues-such as skipped therapy sessions or prolonged inactivity-it generates an alert that is sent directly to the patient and their provider, ensuring timely intervention before therapy effectiveness is compromised. Additionally, the remote monitoring dashboard enables healthcare professionals to track multiple patients, review compliance analytics, and modify treatment plans based on actual usage data. Cloud integration allows for long-term data storage, automatic compliance reporting, and integration with electronic health records (EHRs). By eliminating the need for manual documentation, the system ensures healthcare providers receive reliable, real-time data for therapy evaluation and treatment adjustments.

Thus, in an embodiment the disclosure provides a system, apparatus and a method for monitoring the usage of a therapeutic device by collecting and analyzing electrical consumption and/or vibrational data. The system includes a hardware component designed to measure electrical current flow over time (electrical consumption data) and/or detect inertial and acceleration changes over time (vibrational data) in the therapeutic device; a software component that samples and collects periodic data on changes in electrical current flow and/or vibration from the therapeutic device; a mobile application that utilizes signal processing algorithm and/or machine learning to correlate collected electrical consumption and/or vibrational data with known usage patterns for a specific therapeutic device or a category of therapeutic devices; and a remote monitoring dashboard that displays the therapeutic device's real-time electrical consumption and/or vibration data, identifies activity type, and presents environmental parameters related to device usage.

In an aspect, the disclosure provides a system, apparatus and a method for monitoring the usage of a therapeutic device, wherein the therapeutic device is a cold therapy device that circulates cold water into a brace to reduce temperature, pain, and inflammation in a targeted area of the body.

In another aspect, the disclosure provides a system, apparatus and a method for monitoring the usage of a therapeutic device, wherein the hardware component includes a sensor module comprising a current sensor for measuring electrical consumption and/or an inertial measurement unit (IMU) for detecting vibrational data.

In another aspect, the disclosure provides a system, apparatus and a method for monitoring the usage of a therapeutic device wherein the software component applies a filtering algorithm to remove noise from the electrical consumption and/or vibrational data before analysis.

In another aspect, the disclosure provides a system, apparatus and a method for monitoring the usage of a therapeutic device, wherein the mobile application uses a machine learning model trained on historical usage data from multiple therapeutic devices to recognize and predict usage patterns.

In another aspect, the disclosure provides a system, apparatus and a method for monitoring the usage of a therapeutic device, wherein the remote monitoring dashboard generates real-time alerts for users or caregivers when deviations from expected usage patterns are detected.

In another aspect, the disclosure provides a system, apparatus and a method for monitoring the usage of a therapeutic device, wherein the mobile application includes a secure user authentication feature to protect access to the monitored data.

In another aspect, the disclosure provides a system, apparatus and a method for monitoring the usage of a therapeutic device, wherein the remote monitoring dashboard provides information on battery life, maintenance alerts, and the operational status of the therapeutic device.

In another aspect, the disclosure provides a system, apparatus and a method for monitoring the usage of a therapeutic device, wherein the software component includes an adaptive learning feature that refines known usage patterns over time based on user-specific behavior.

In another aspect, the disclosure provides a system, apparatus and a method for monitoring the usage of a therapeutic device, wherein data collected by the system is transmitted to a remote cloud server for long-term storage and further analysis.

In another aspect, the disclosure provides a system, apparatus and a method for monitoring the usage of a therapeutic device, wherein the system integrates with third-party healthcare monitoring platforms to provide a comprehensive overview of therapy adherence.

FIG. 1 illustrates an exemplary embodiment of a system for detecting usage of a therapeutic device from its electricity consumption and/or vibration showing the apparatus, device and power connection setup. As shown in this figure, the apparatus can be implemented in multiple ways, including a system having an electrical outlet 1, a power cord 2, which can be embedded with an apparatus 3, which can also include a current sensor and/or a vibration sensor connected to a therapeutic device 4. This configuration enables the system to track when the device is plugged in, monitor power consumption and/or vibrational trends, and detect operational anomalies.

FIG. 2 illustrates another exemplary embodiment of a system for detecting usage of a therapeutic device from its electricity consumption and/or vibration showing the apparatus, device and power connection setup. As shown in this figure, the apparatus is shown as a plug-in adapter 3, which connects between the wall outlet 1, the power cord 2, and the therapeutic device 4, allowing it to capture electrical and/or vibrational consumption data.

FIG. 3 illustrates yet another exemplary embodiment of a system for detecting usage of a therapeutic device from its electricity consumption and/or vibration showing the apparatus, device and power connection setup. As shown in this figure, this involves an apparatus 3, which attaches directly to the therapeutic device 4. This approach is particularly effective for battery-powered devices, where direct electrical monitoring is not feasible.

Additional implementations of the hardware component of the apparatus may include multi-sensor fusion, where gyroscopes, accelerometers, magnetometers, and piezoelectric sensors work in combination to enhance vibrational data accuracy. In some embodiments, the system may incorporate temperature sensors, humidity sensors, or air pressure sensors to provide additional environmental context for therapy monitoring. These expanded sensor options allow for more precise data analysis and broader compatibility across different therapeutic devices.

FIG. 4 illustrates an exemplary embodiment of the software components of the system for detecting usage of a therapeutic device from its electricity consumption and/or vibration, which can operate in two primary modes: a learning mode and an operational mode. As shown in this figure, the system 100 includes a therapeutic device 100 and an apparatus 200. With the therapeutic device 100, a user can move from a start 101 position, a power on 102 position, a select compression 103 position, a select temperature 104 position, a set time option 105 position, and a start therapy 106 position. Alternatively, these settings may be pre-set 107. After therapy, the user can move to the end therapy 108 position, the power off 109 position, and the end 110 position.

The process begins with the system initialization at the start phase, marking the beginning of the therapy setup. Once initiated, the device is powered on, activating its system and preparing it for user configuration. At this stage, the user selects the appropriate compression setting, adjusting the pressure levels to match the desired therapy requirements. Following this, the temperature is selected, ensuring the optimal heat level is set for the session. To complete the initial setup, the user specifies the session duration by setting a timer, allowing the therapy to proceed for a predefined period.

With all pre-set configurations in place, the system transitions into the therapy phase, where the therapy session officially begins. The device operates according to the specified parameters, applying the selected compression and temperature settings for the predetermined time. As the session progresses, the therapy is delivered continuously until the timer reaches completion, signaling the end of the session. Once the therapy session concludes, the system enters the shutdown phase, where the device is powered off to ensure all active functions cease operation. This step prevents unnecessary energy consumption and ensures the system is safely disengaged. Finally, the process formally ends, completing the therapy cycle.

As shown in this figure, the apparatus 200 includes an electrical and/or vibrational monitoring 202 position, followed by a learn mode 203 or an operational mode 204. In the learn mode 203, electrical and/or vibrational data is acquired and stored 205, which may be repeated 206 as needed. Next, the data pattern is analyzed 207 and a device usage profile is created for each operational state. Data can then be stored for future use by an operational algorithm 208 or can be stored in a database 210.

The software component of the system operates in two primary modes: learning mode and operational mode. During learning mode, the system records baseline usage data to establish a reference profile of how the device should operate under normal conditions. This baseline includes power consumption signatures, expected vibration frequencies, therapy duration parameters, and device-specific thresholds. Once this profile is created, the system transitions into operational mode, where it continuously compares real-time data to the baseline profile.

As shown in this figure, while in the operational mode 204, electrical and/or vibrational signals are acquired, which includes applying an algorithm, using stored data to characterize real time machine profile 212. Next, a display provides user friendly information on real time usage 213, which can be transferred via the internet to a smart phone 214 or the data can be stored for dashboard development 215, as well as in the database 210.

Beyond its core functionality, the system incorporates a monitoring and data processing phase that enhances its effectiveness through real-time tracking and analysis. During the therapy session, the system continuously monitors electrical and vibration signals, gathering crucial data to assess operational efficiency. As part of this monitoring, the system evaluates whether to operate is in learning mode or operational mode, determining the most suitable approach based on the collected data.

If the system determines that additional data collection is needed, it enters learning mode, where operational data is actively gathered for further optimization. In this phase, the device acquires and stores electrical signals generated during therapy sessions. Once collected, these signals are analyzed to identify patterns and create a comprehensive power usage profile for different operational states. This information is invaluable in refining the system's efficiency, and it is stored for future reference, contributing to the development of improved operational algorithms. If necessary, this learning process is repeated multiple times until a sufficient dataset is acquired, ensuring the system has enough information for continuous improvement.

Alternatively, when the system is functioning under operational mode, it actively acquires real-time signals and applies pre-stored algorithms to enhance its performance. Using this real-time data, the system characterizes the device's current operational profile, ensuring that it operates within optimal parameters. To improve user experience, the system presents this information in an intuitive, user-friendly format, providing real-time feedback on the therapy session's progress. Additionally, all relevant data collected during therapy is stored in a centralized database, allowing for further analysis and dashboard development. This storage ensures that the system can continuously refine its algorithms, leveraging past data to optimize future therapy sessions.

By integrating real-time monitoring, adaptive learning, and user-friendly feedback, this structured approach ensures that therapy sessions are both effective and continuously improving, leveraging intelligent data-driven optimization to enhance user experience and system efficiency.

If an anomaly is detected, such as an unexpected power surge, inconsistent vibrations, or an incomplete therapy session, the system generates an alert and transmits the data to the mobile application and remote dashboard. The machine learning algorithm continuously improves its anomaly detection capabilities by analyzing historical data stored in the usage database, reducing false positives while improving therapy tracking accuracy.

The wireless communication module transmits collected data to the cloud-based analytics platform, where it is stored for long-term compliance tracking and historical trend analysis. The cloud system supports electronic health record (EHR) integration, allowing healthcare providers to seamlessly access device usage data alongside other patient records.

A key feature of the cloud system is its predictive analytics capability. By analyzing historical usage patterns, the system can predict therapy adherence trends and anticipate non-compliance issues before they occur. For example, if a patient frequently skips therapy sessions at a particular time of day, the system may recommend customized reminders or alternative scheduling. If a device shows signs of potential failure—such as declining motor efficiency or erratic power consumption—the system can notify technicians for preventive maintenance, reducing the likelihood of device downtime.

A method of using the system begins with installing the hardware component onto the therapeutic device, whether as a power cord integration, plug-in adapter, or external sensor module. Once activated, the system enters learning mode, establishing a baseline operation profile. It then transitions to operational mode, where it monitors and analyzes electrical and vibrational data in real-time. If irregularities are detected, the system generates alerts, transmits data to the cloud, and provides real-time feedback through the mobile application and dashboard.

FIG. 5 illustrates an exemplary embodiment of a system for detecting usage of a therapeutic device, e.g., a cold therapy machine, from its electricity consumption and/or vibration. As shown in this figure, the system is highly effective for monitoring cold therapy machines. A core device connects Bluetooth to the ice device, wherein a mobile application monitors the patients usage/adherence and data. The mobile application provides behavioral feedback, positive reinforcement and alerts in case of nonadherence. Each event is recorded enabling data driven interaction, where clinicians are notified if the patient is able/unable to comply. By continuously tracking electrical consumption, cooling cycles, and vibrational activity, it ensures that therapy is being delivered consistently and effectively. If a patient turns off the device prematurely, skips a session, or experiences a device malfunction, the system logs these deviations and sends a notification to both the patient and their healthcare provider. Over time, this data allows clinicians to review compliance trends, assess therapy effectiveness, and refine treatment plans based on real-world insights.

The system consists of a cold therapy device integrated with a wireless sensor module, a mobile application, a cloud-based data processing system, and a clinician dashboard. The wireless sensor module connects to the cold therapy device and transmits real-time usage data, such as duration and temperature, via Bluetooth to the mobile application. The mobile application records and analyzes patient adherence patterns, offering immediate feedback through positive reinforcement messages and alerts in cases of non-adherence. Each recorded event is uploaded to a secure cloud platform, where data is processed and stored for review.

The cloud-based system enables healthcare providers to access adherence data remotely through an interactive clinician dashboard. This dashboard presents real-time and historical data, allowing clinicians to track patient compliance and intervene if necessary. By leveraging wireless communication and data analytics, the system ensures accurate monitoring without reliance on self-reported compliance. The integration of automated feedback mechanisms enhances patient engagement and encourages consistent use of the cold therapy device.

The advantages of this system include automated adherence tracking, behavioral reinforcement, remote monitoring for clinicians, and seamless wireless data transmission. The ability to analyze adherence trends and provide timely feedback improves patient outcomes by ensuring proper use of the therapy device. This innovation offers a comprehensive solution for improving compliance with cold therapy treatments through real-time monitoring, cloud-based analytics, and proactive clinician involvement.

By integrating passive monitoring, real-time alerts, and cloud-based predictive analytics, this system ensures therapy adherence, enhances patient outcomes, and improves healthcare provider decision-making. Its scalability and adaptability make it suitable for a wide range of therapeutic devices, including cold therapy units, electrical stimulation devices, compression therapy systems, and wearable rehabilitation equipment. This intelligent monitoring system revolutionizes patient compliance tracking, providing an automated, data-driven approach to ensure medical devices are used as intended, therapy plans are followed accurately, and patients receive the most effective treatment possible.

What is claimed is:

1. A system for monitoring the usage of a therapeutic device by a user by an apparatus that monitors electrical consumption and/or vibrational data, comprising:
a hardware component designed to measure electrical current flow over time (electrical consumption data) and/or detect inertial and acceleration changes over time (vibrational data) in the therapeutic device;
a software component that samples and collects periodic data on changes in electrical current flow and/or vibration from the therapeutic device;
a mobile application that utilizes signal processing algorithm and/or machine learning to correlate collected electrical consumption and/or vibrational data with known usage patterns for a specific therapeutic device or a category of therapeutic devices; and
a remote monitoring dashboard that displays the therapeutic device's real-time electrical consumption and/or vibration data, identifies activity type, and presents environmental parameters related to device usage.

2. The system of claim 1, wherein the therapeutic device is a cold therapy device that circulates cold water into a brace to reduce temperature, pain, and inflammation in a targeted area of the body.

3. The system of claim 1, wherein the hardware component includes a sensor module comprising a current sensor for measuring electrical consumption and an inertial measurement unit (IMU) for detecting vibrational data.

4. The system of claim 1, wherein the software component applies a filtering algorithm to remove noise from the electrical consumption and/or vibrational data before analysis.

5. The system of claim 1, wherein the mobile application uses a machine learning model trained on historical usage data from multiple therapeutic devices to recognize and predict usage patterns.

6. The system of claim 1, wherein the remote monitoring dashboard generates real-time alerts for users or caregivers when deviations from expected usage patterns are detected.

7. The system of claim 1, wherein the mobile application includes a secure user authentication feature to protect access to the monitored data.

8. The system of claim 1, wherein the remote monitoring dashboard provides information on battery life, maintenance alerts, and the operational status of the therapeutic device.

9. The system of claim 1, wherein the software component includes an adaptive learning feature that refines known usage patterns over time based on user-specific behavior.

10. The system of claim 1, wherein data collected by the system is transmitted to a remote cloud server for long-term storage and further analysis.

11. The system of claim 1, wherein the system integrates with third-party healthcare monitoring platforms to provide a comprehensive overview of therapy adherence.
